(19) United States
(12) Patent Application Publication
(10) Pub. No.: US 2023/0326079 A1
(43) Pub. Date: Oct. 12, 2023

(54) CALIBRATION DEVICE, ENDOSCOPE SYSTEM, CALIBRATION METHOD, AND COMPUTER-READABLE RECORDING MEDIUM

(71) Applicant: OLYMPUS CORPORATION, Tokyo (JP)

(72) Inventor: Kazuhito HORIUCHI, Tokyo (JP)

(73) Assignee: OLYMPUS CORPORATION, Tokyo (JP)

(21) Appl. No.: 18/208,318

(22) Filed: Jun. 12, 2023

Related U.S. Application Data
(63) Continuation of application No. PCT/JP2021/023829, filed on Jun. 23, 2021.

(51) Int. Cl. G06T 7/80; A61B 1/00

(52) U.S. Cl. CPC G06T 7/85; A61B 1/00193; A61B 1/00057; A61B 1/0005; G06T 2207/10028; G06T 2207/10068

(57) ABSTRACT

A calibration device includes a processor configured to process a stereo image which is taken by a stereo camera disposed in front end portion of an endoscope, the processor being configured to superimpose an imaging guide onto the stereo image to generate a calibration image that is to be displayed in a display, and calibrate a camera parameter of the stereo camera based on the stereo image taken by the stereo camera is state in which a plurality of marks included is a chart representing a photographic subject image in the calibration image fall into a specific positional relationship with respect to the imaging guide.

### CROSS-REFERENCE TO RELATED APPLICATION

This application is a continuation of International Application No. PCT/JP2021/023829, filed on Jun. 23, 2021, the entire contents of which are incorporated herein by reference.

### BACKGROUND

### 1. Technical Field

The present disclosure relates to a calibration device, an endoscope system, a calibration method, and a computer-readable recording medium.

### 2. Related Art

In the related art, the stereo measurement technology is known as one of the distance measurement technologies meant for measuring the distance to a photographic subject that represents the target for imaging. In the stereo measurement technology, simultaneous imaging from different viewpoints is performed using a stereo camera; and, using the relative deviation of the same photographic subject among the images, the three-dimensional position of the photographic subject is calculated based on the triangulation principle. In order to calculate the three-dimensional position of the photographic subject, the camera parameters of the stereo camera need to be available. As far as the camera parameters are concerned, internal parameters can be cited that include the focal length and the distortion coefficient of the lens installed in the stereo camera, and include the central position of the optical axis of the lens in the images; and external parameters can be cited that indicate the positional relationship and the pose relationship among stereo cameras.

For example, in the technology disclosed in Japanese Patent Application Laid-open No. 2017-003279, imaging of a chart is performed using a stereo camera; and, based on the stereo image that is obtained, the camera parameters are calculated (calibrated). That chart has a plurality of marks formed therein.

### SUMMARY

In some embodiments, a calibration device includes a processor configured to process a stereo image which is taken by a stereo camera disposed in front end portion of an endoscope, the processor being configured to superimpose an imaging guide onto the stereo image to generate a calibration image that is to be displayed in a display, and calibrate a camera parameter of the stereo camera based on the stereo image taken by the stereo camera in state in which a plurality of marks included in a chart representing a photographic subject image in the calibration image fall into a specific positional relationship with respect to the imaging guide.

In some embodiments, an endoscope system includes: an endoscope that includes a stereo camera installed at front end portion of the endoscope; a calibration device that includes a processor configured to process a stereo image taken by the stereo camera; and a display configured to display an image, the processor being configured to superimpose an imaging guide onto the stereo image to generate a calibration image that is to be displayed in the display, and calibrate a camera parameter of the stereo camera based on the stereo image taken by the stereo camera in state in which a plurality of marks included in a chart representing a photographic subject image in the calibration image fall into a specific positional relationship with respect to the imaging guide.

In some embodiments, provides is a calibration method implemented by a processor of a calibration device. The calibration method includes: superimposing an imaging guide onto a stereo image which is taken by a stereo camera disposed in front end portion of an endoscope to generate a calibration image that is to be displayed in a display; and calibrating a camera parameter of the stereo camera based on the stereo image taken by the stereo camera in state in which a plurality of marks included in a chart representing a photographic subject image in the calibration image fall into a specific positional relationship with respect to the imaging guide.

In some embodiments, provided is a non-transitory computer-readable recording medium with an executable program stored thereon. The program causes a processor of a calibration device to execute: superimposing an imaging guide onto a stereo image which is taken by a stereo camera disposed in front end portion of an endoscope to generate a calibration image that is to be displayed in a display; and calibrating a camera parameter of the stereo camera based on the stereo image taken by the stereo camera in state in which a plurality of marks included in a chart representing a photographic subject image in the calibration image fall into a specific positional relationship with respect to the imaging guide.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a configuration of an endoscope system according to an embodiment;

FIGS. 2 to 9 are diagrams for explaining a calculation method for calculating the three-dimensional position of a photographic subject;

FIG. 10 is a diagram illustrating a configuration of a chart;

FIG. 11 is a flowchart for explaining a calibration method;

FIG. 12 is a diagram illustrating a calibration image displayed in a display device;

FIG. 13 is a diagram illustrating a first modification example of the embodiment;

FIG. 14 is a diagram illustrating a second modification example of the embodiment;

FIG. 15 is a diagram illustrating a third modification example of the embodiment;

FIG. 16 is a diagram illustrating a fourth modification example of the embodiment;

FIG. 17 is a diagram illustrating a fifth modification example of the embodiment;

FIG. 18 is a diagram illustrating a sixth modification example of the embodiment;

FIG. 19 is a diagram illustrating a seventh modification example of the embodiment;

FIG. 20 is a diagram illustrating an eighth modification example of the embodiment;

FIG. 21 is a diagram illustrating a ninth modification example of the embodiment;

FIG. 22 is a diagram illustrating a 10-th modification example of the embodiment; and

FIG. 23 is a diagram illustrating an 11-th modification example of the embodiment.

### DETAILED DESCRIPTION

An illustrative embodiment (hereinafter, embodiment) of the disclosure is described below with reference to the accompanying drawings. However, the disclosure is not limited by the embodiment described below. Meanwhile, in the drawings, identical constituent elements are referred to by the same reference numerals.

### Configuration of Endoscope System

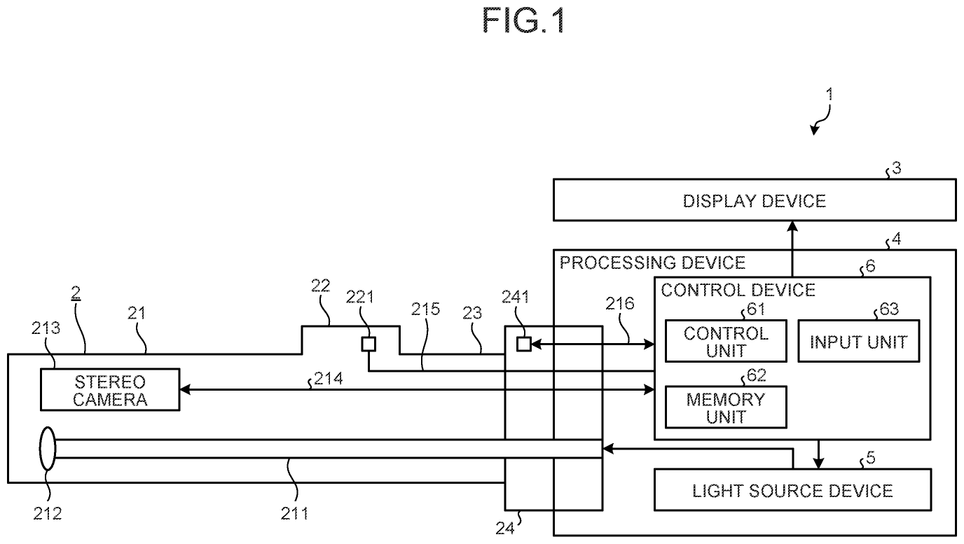

An endoscope system 1 is used, for example, in the medical field. In the endoscope system 1, while observing the inside of a subject (in vivo), the three-dimensional position of a photographic subject in vivo is calculated based on the triangulation principle. As illustrated in FIG. 1, the endoscope system 1 includes an endoscope 2, a display device 3, and a processing device 4.

The endoscope 2 is partially inserted in vivo; performs imaging of a photographic-subject image formed as a result of reflection occurring in vivo; and outputs an image signal that is generated as a result of performing imaging. As illustrated in FIG. 1, the endoscope 2 includes an insertion portion 21, an operating unit 22, universal cord 23, and a connector unit 24.

The insertion portion 21 is at least partially flexible and gets inserted in vivo. In the insertion portion 21 are installed a light guide 211, an illumination lens 212, a stereo camera 213, and signal lines 214 to 216.

The light guide 211 is drawn from the insertion portion 21 up to the connector unit 24 via the operating unit 22 and the universal cord 23. One end of the light guide 211 is positioned at the front end portion of the insertion portion 21. In the state in which the endoscope 2 is connected to the processing device 4, the other end of the light guide 211 is positioned inside the processing device 4. Then, the light guide 211 transmits the light, which is supplied from a light source device 5 of the processing device 4, from the concerned end to the front end.

In the insertion portion 21, the illumination lens 212 faces the front end of the light guide 211. Then, the illumination lens 212 bombards, in vivo, the light transmitted by the light guide 211.

The stereo camera 213 is disposed at the front end portion of the insertion portion 21. The stereo camera 213 takes simultaneous images of the photographic subject from different directions, and generates a stereo image including a plurality of images having a mutual parallax.

For example, in order to generate a plurality of images (a stereo image), the stereo camera 213 includes a single imaging sensor such as a charge coupled device (CCD) or a complementary metal oxide semiconductor (CMOS). In that case, the images are equivalent to images in a plurality of imaging regions set on the imaging face of the single imaging sensor. Alternatively, as the stereo camera 213, it is possible to use a plurality of imaging sensors each of which generates a plurality of images. In the present embodiment, the stereo camera 213 generates a stereo image that includes a right-side image and a left-side image having a parallax in the right-left direction. Meanwhile, the stereo camera 213 is not limited to be configured to generate a stereo image that includes two images (a right-side image and a left-side image). Alternatively, the stereo camera 213 can be configured to generate a stereo image that includes three or more images.

As far as the configuration of the stereo camera 213 is concerned, for example, it is possible to adopt the configuration of the stereo camera as disclosed in International Laid-open Pamphlet No. 2019/087253.

The signal line 214 is drawn from the insertion portion 21 up to the connector unit 24 via the operating unit 22 and the universal cord 23. One end of the signal line 214 is electrically connected to the stereo camera 213. In the state in which the endoscope 2 is connected to the processing device 4, the other end of the signal line 214 is electrically connected to a control device 6 of the processing device 4. The signal line 214 transmits the control signals that are output from the control device 6, and transmits the stereo images (image signals) that are output from the stereo camera 213.

The operating unit 22 is connected to the proximal end portion of the insertion portion 21. The operating unit 22 receives a variety of operations intended for the endoscope 2. For example, in the operating unit 22, a release button 221 is installed (see FIG. 1) that receives an operation for incorporating a stereo image as a still image.

The signal line 215 is drawn from the operating unit 22 up to the connector unit 24 via the universal cord 23. One end of the signal line 215 is electrically connected to the release button 221. In the state in which the endoscope 2 is connected to the processing device 4, the other end of the signal line 215 is electrically connected to the control device 6 of the processing device 4. The signal line 215 transmits an operation signal in response to an operation performed with respect to the release button 221.

The universal cord 23 extends from the operating unit 22 in a direction different than the direction of extension of the insertion portion 21, and has the light guide 211 and the signal lines 214 and 215 disposed thereon.

The connector unit 24 is disposed at an end portion of the universal cord 23, and is connected to the processing device 4 in a detachably-attachable manner. The connector unit 24 has a memory unit 241 installed therein (see FIG. 1) The memory unit 241 is used to store a scope identifier (ID) meant for uniquely identifying the endoscope 2.

One end of the signal line 216 is electrically connected to the memory unit 241. In the state in which the endoscope 2 is connected to the processing device 4, the other end of the signal line 216 is electrically connected to the control device 6 of the processing device 4. The signal line transmits a signal according to the scope ID stored in the memory unit 241.

The display device 3 is a liquid crystal display (LCD) or an electro luminescence (EL) display that is used to display images with respect to which image processing has been performed by the processing device 4.

As illustrated in FIG. 1, the processing device 4 includes the light source device 5 and the control device 6. In the present embodiment, the light source device 5 and the control device 6 are disposed inside a single housing as the processing device 4. However, that is not the only possible case. Alternatively, the light source device 5 and the control device 6 can be disposed in separate housings.

Under the control of the control device 6, the light source device 5 supplies a specific illumination light to the other end of the light guide 211.

The control device 6 is equivalent to a calibration device according to the disclosure. The control device 6 comprehensively controls the operations of the entire endoscope system 1. As illustrated in FIG. 1, the control device 6 includes a control unit 61, a memory unit 62, and an input unit 63.

The control unit 61 is equivalent to a processor according to the disclosure. The control unit 61 is configured using a central processing unit (CPU) or a field-programmable gate array (FPGA), and controls the operations of the entire endoscope system 1 according to the computer programs stored in the memory unit 62. Regarding the functions of the control unit 61, the explanation is given later in sections “calculation method for calculating three-dimensional position of photographic subject” and “calibration method”.

The memory unit 62 is used to store various computer programs (including a calibration program according to the disclosure) that are executed by the control unit 61, and to store the information required in the operations performed by the control unit 61.

Examples of the information required in the operations performed by the control unit 61 include associated information in which the scope ID is associated to the camera parameters of the stereo camera 213 that constitutes the endoscope 2.

The input unit 63 is configured using a keyboard, a mouse, a switch, or a touch-sensitive panel; and receives user operations. Then, in response to a user operation, the input unit 63 outputs an operation signal to the control unit 61.

Calculation method for calculating three-dimensional position of photographic subject

Given below is the explanation about a calculation method for calculating the three-dimensional position of the photographic subject as implemented in the control unit 61.

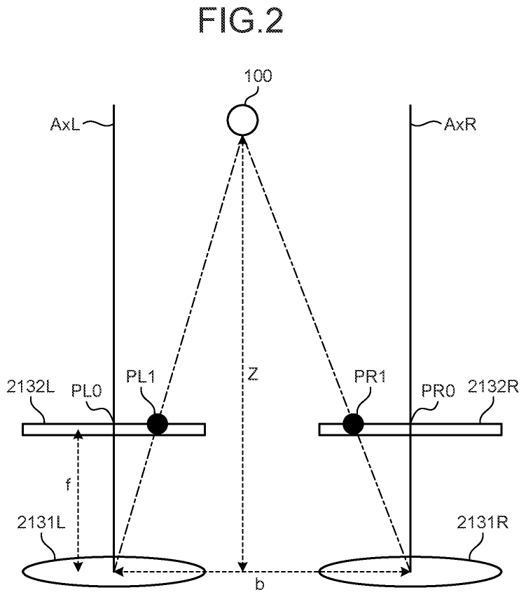

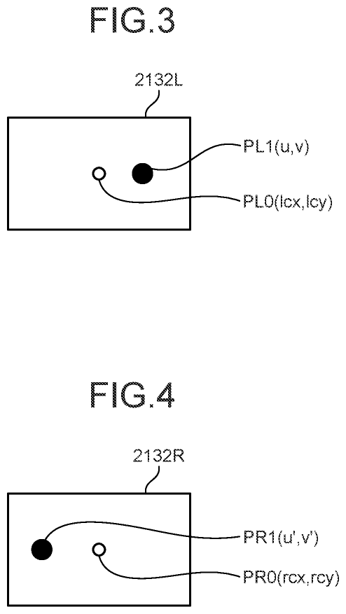

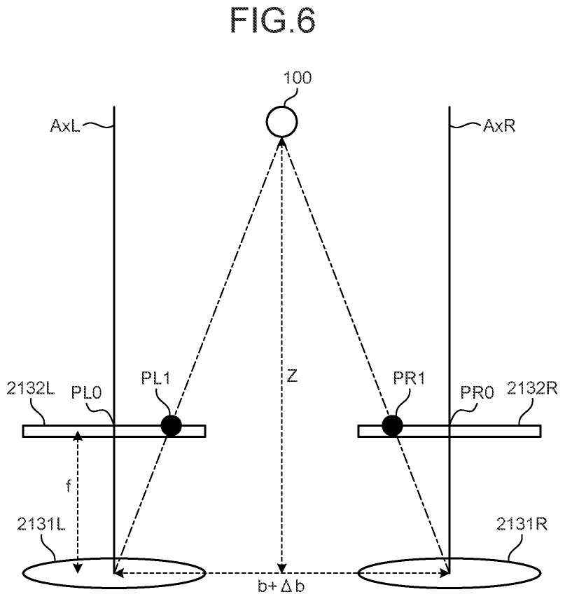

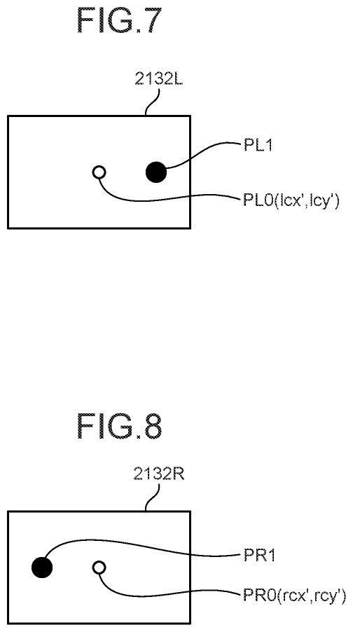

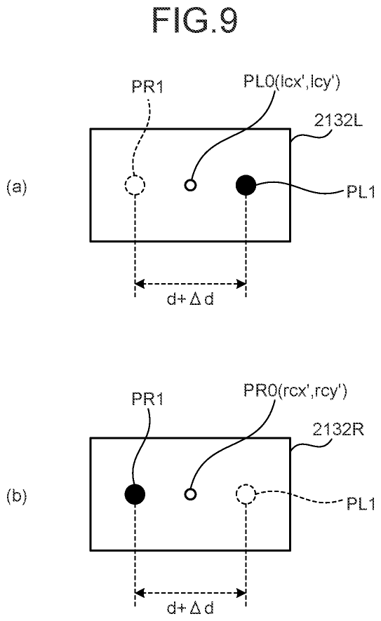

FIGS. 2 to 9 are diagrams for explaining the calculation method for calculating the three-dimensional position of photographic subject 100. In FIGS. 2 and 6, a left-side optical system 2131L and a right-side optical stem 2131R are illustrated that constitute the stereo camera 213. Moreover, an optical axis AxL represents the optical axis of the left-side optical system 2131L, and an optical axis AxR represents the optical axis of the right-side optical system 2131R. In FIGS. 2 to 4 and in FIGS. 6 to 8, a left-side imaging face (perspective projection) 2132L and a right-side imaging face (perspective projection) 2132R are illustrated that constitute the stereo camera 213. The left-side imaging face 2132L is equivalent to a left-side image taken the stereo camera 213, and the right-side imaging face 2132R is equivalent to a right-side image taken by the stereo camera 213. The left-side imaging face 2132L and the right-side imaging face 2132R either can be installed in a single imaging sensor as explained earlier, or can be configured as imaging faces of two imaging sensors.

Herein, assume at b [mm] represents the baseline length in the form of the distance between the optical axes AxL and AxR; f [mm] represents the focal length of the stereo camera 213; and δ [mm/pixel] represents the pixel pitch of the imaging sensor constituting the stereo camera 213.

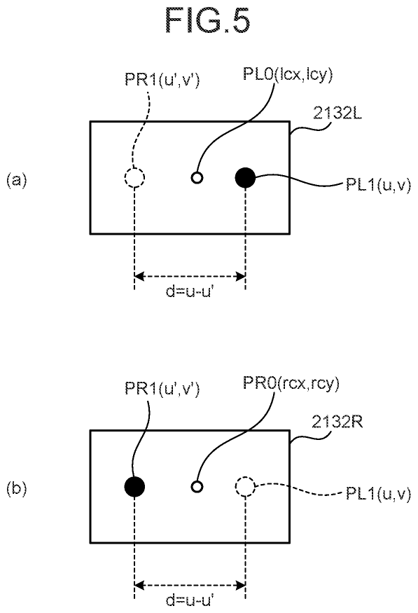

Regarding the photographic subject 100 present at a distance Z [mm] from the stereo camera 213, an image is formed at a position PL1 on the left-side imaging face 2132L and an image is formed at a position PR1 on the right-side imaging face 2132R. A. coordinate position (u, v) of the position PL1 represents the coordinate position when a point that is present in the left-side imaging face 2132L and present on the optical axis AxL of the left-side optical system 2131L is treated as an origin PL0 (having a coordinate position (lcx, lcy)). In an identical manner, a coordinate position (u′, v′) of the position PR1 represents the coordinate position when a point that is present in the right-side imaging face 2132R and present on the optical axis AxR of the right-side optical system 2131R is treated as an origin PR0 (having a coordinate position (rcx, rcy)). Then, a difference u-u′ between the image formation positions of the same photographic subject on the left-side imaging face 2132L and the right-side imaging face 2132R represents a parallax d [pixel] (see FIG. 5).

Based on the triangulation principle, the control unit 61 calculates the distance Z to the photographic subject according to Equation 1 given below.

$$\begin{matrix}Z = \frac{b}{\frac{δ u}{f} - \frac{δ u}{f}} \\ = \frac{fb}{δ ( u - {u}^{′} )} \\ = \frac{fb}{δ d}\end{matrix} \qquad ( 1 )$$

The pixel pitch δ is a known value decided according to the specifications of the imaging sensor constituting the stereo camera 213. The coordinate positions (lcx, lcy) and (rcx, rcy) of the origins PL0 and PR0, respectively, associated to the baseline length b, and the focal length f are decided in advance as the internal parameters of a camera according to a known calibration algorithm (for example, Zhengyou Zhang, “A Flexible New Technique for Camera Calibration”, IEEE TRANSACTIONS ON PATTERN ANALYSIS AND MACHINE INTELLIGENCE, VOL. 22, No. 11, NOVEMBER 2000, 1330-1334). Then, the pixel pitch δ, the baseline length b, the coordinate position (lcx, lcy) of the origin PL0, the coordinate position (rcx, rcy) of the origin PR0, and the focal length f are stored in advance, in the memory unit 62, as the camera parameters of the stereo camera 213 in a corresponding manner to the concerned scope ID.

With an increase in the period of time of using the endoscope 2, due to the temporal changes occurring in the positions of the left-side optical system 2131L and the right-side optical system 2131R, the baseline length b and the focal length f that are calculated according to the abovementioned calibration algorithm also go on changing. The variation in the baseline length b and the focal length f gets reflected in the parallax d, which in turn affects the calculation of the distance Z.

Particularly, as compared to the variation in the focal length f, the variation in the baseline length b gets reflected more in the parallax d. FIGS. 6 to 9 are diagrams which correspond to FIGS. 2 to 5, respectively, and in which the baseline length b has shifted by an amount Δb. For example, in the state in which of having the baseline length b+Δb, if the parallax at the distance Z [mm] is equal to d+Δd, then the distance Z [mm] can be obtained according to Equation 2 given below instead of Equation 1 given earlier.

$$Z = \frac{f ( b + Δ b )}{δ ( d + Δ d )} \qquad ( 2 )$$

However, since there is no way for the user to confirm the variation in the baseline length, the calculation of the distance is carried out according to the pre-temporal-change state (i.e., using the baseline length b, the focal length f, and the pixel pitch δ). A distance Z′ [mm] that is calculated at that time is given below in Equation 3. The distance Z′ [mm] happens to be different than the actual distance Z [mm] given above in Equation 2.

$${Z}^{′} = \frac{fb}{δ ( d + Δ d )} \qquad ( 3 )$$

Till now, the focus was only on the distance Z [mm] to the photographic subject 100. However, a three-dimensional position (X, Y, Z) [mm] of the photographic subject also gets affected by the variation in the baseline length b.

More particularly, based on the triangulation principle, the control unit 61 calculates the X and Y coordinates according to Equations 4 and 5 given below. The coordinates X and Y are calculated with the optical axis center of the left-side optical system 2131L serving as the coordinate origin of the three-dimensional space.

$$\begin{matrix}X = \frac{Z ( δ u )}{f} \\ = \frac{bu}{d}\end{matrix} \qquad ( 4 )$$

$$\begin{matrix}Y = \frac{Z ( δ v )}{f} \\ = \frac{bv}{d}\end{matrix} \qquad ( 5 )$$

As given in Equations 4 and 5, the baseline length b is dependent on the coordinates X and Y. Hence, if the baseline length b undergoes any temporal change, it no more becomes possible to correctly calculate the three-dimensional position (X, Y, Z) of the photographic subject 100.

In the present embodiment, it is the baseline length that represents the camera parameter which affects the calculation accuracy of the three-dimensional position (X, Y, Z). Hence, only the baseline length is calibrated in order to correct the calculation accuracy. In that calibration, a chart 200 given below is used.

### Configuration of Chart

FIG. 10 is a diagram illustrating a configuration of a chart 200.

The chart 200 is a pattern formed on the outer surface of a translucent material such as a glass having a circular shape in the planar view.

More particularly, on the outer surface of the chart 200, as illustrated in FIG. 10, two marks 201 and 202 are formed across the center position of the chart 200. The two marks 201 and 202 have an identical circular shape, are white in color, and are placed to have the inter-centroid distance therebetween to be equal to a known value of D [mm].

On the outer surface of the chart 200, the region other than the two marks 201 and 202 is black in color. In FIG. 10, the black color is expressed in the form of hatched lines.

Meanwhile, the chart 200 is not limited to be made of glass as mentioned above. Alternatively, the chart 200 can be displayed on the screen of a tablet.

### Calibration Method

Given below is the explanation of a calibration method implemented by the control unit 61 for calibrating the camera parameters.

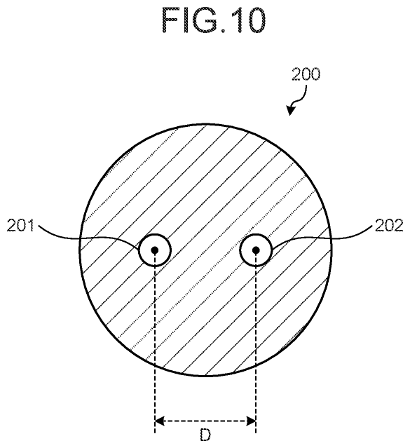

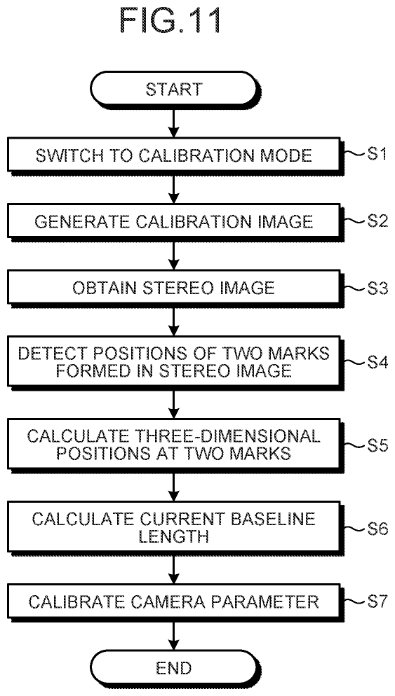

FIG. 11 is a flowchart for explaining the calibration method.

Firstly, the control unit 61 switches the endoscope system 1 to a calibration mode (Step S1). In the calibration mode, calibration image F is generated as well as the camera parameters are corrected. Other than the calibration mode, the endoscope system 1 has an observation mode in which the operations of the endoscope 2 are controlled for observing in vivo.

After the operation at Step S1 is performed, the control unit 61 generates the calibration image F and displays it in the display device 3 (Step S2).

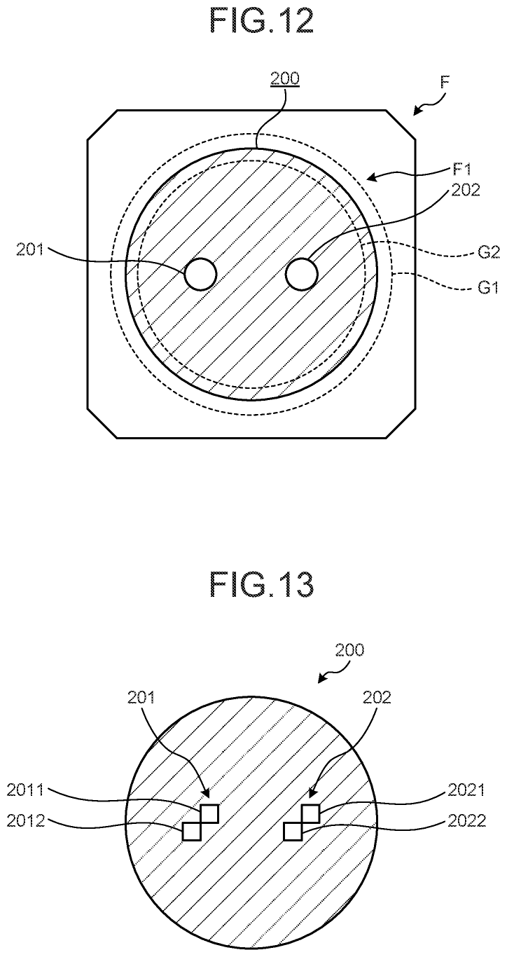

FIG. 12 is a diagram illustrating the calibration image F displayed in the display device 3. In FIG. 12, for explanatory convenience, it is illustrated that the chart 200 is included as a photographic subject image in the left-side image (hereinafter, referred to as a main image F1) taken by the stereo camera 213.

More particularly, between the left-side image and the right-side image taken by the stereo camera 213, the control unit 61 treats the left-side image as the main image F1, and performs predetermined image processing with respect to the main image F1. Examples of the image processing include optical black subtraction, white balance adjustment, de-mosaic processing, color correction matrix processing, gamma correction, and YC processing in which RGB signals (normal optical images) are converted into luminance/color difference signals (Y, Cr/Cb signals). Then, the control unit 61 generates the calibration image F by superimposing imaging guides G1 and G2 (see FIG. 12) onto the main image F1 that has been subjected to predetermined image processing, and displays the calibration image F in the display device 3. Meanwhile, the calibration image F is displayed in the live state.

As illustrated in FIG. 12, the imaging guide G1 is circular in shape and represents a guide for positioning the two marks 201 and 202 in the central region of the calibration image F.

The imaging guide G2 is circular in shape and is smaller in size than the imaging guide G1, and represents a guide for ensuring that the two marks 201 and 202 are separated by a specific gap therebetween in the calibration image F. In other words, the imaging guide G2 represents a guide for ensuring that the two marks 201 and 202 have specific depth positions.

While keeping the front end of the insertion portion 21 directed toward the chart 200, the operator confirms the calibration image F displayed in the display device 3. Moreover, while confirming the calibration image F, the operator operates the insertion portion 21 so as to position the two marks 201 and 202 within the imaging guide G1 and to position the outer edge of the chart 200 in between the imaging guides G1 and G2 (i.e., the state illustrated in FIG. 12). Once the state illustrated in FIG. 12 is set, the operator presses the release button 221. As a result, the control unit 61 obtains the stereo image (i.e., the left-side image (the main image F1) and the right-side image) taken by the stereo camera 213 at the time when the release button 221 was pressed (Step S3). In other words, of the calibration image F that was being displayed in the display device at the time when the release button 221 was pressed, the control unit 61 obtains the main image F1 in which the imaging guides G1 and G2 are not displayed and obtains the right-side image taken by the stereo camera 213 at the time when the release button 221 was pressed. The main image F1 and the right-side image are the images taken by the stereo camera 213 when the two marks 201 and 202 fall into a specific positional relationship with respect to the imaging guides G1 and G2. Meanwhile, the main image F1 and the right-side image are subjected to the image process explained earlier, and are eligible for the subsequent processing.

After the operation at Step S3 is performed, the control unit 61 detects the positions of the two marks 201 and 202 included in the main image F1 and the right-side image that are obtained at Step 53 (Step S4).

More particularly for example, the control unit 61 performs binarization with respect to that region in the main image F1 in which only the chart 200 is included. As a result, the control unit 61 recognizes the two marks 201 and 202 included in the main image F1. Then, the control unit 61 calculates the centroid position of the mark 201, and treats the centroid position as the position of the mark 201. In an identical manner, the control unit 61 calculates the centroid position of the mark 202, and treats the centroid position as the mark 202. Moreover, regarding the two marks 201 and 202 included in the right-side image obtained at Step S3, the positions are detected in an identical manner.

In the following explanation, a coordinate position (u1L, v1L) represents the coordinate position of the mark 201 included in the main image F1 and detected at Step S4. Moreover, coordinate position (u2L, v2L) represents the coordinate position of the mark 202 included in the main image F1 and detected at Step S4. Furthermore, a coordinate position (u1R, v1R) represents the coordinate position of the mark 201 included in the right-side image and detected at Step S4. Moreover, a coordinate position (u2R, v2R) represents the coordinate position of the mark 202 included in the right-side image and detected at Step S4.

After the operation at Step S4 is performed, the control unit 61 calculates the three-dimensional positions of the two marks 201 and 202 based on the triangulation principle (Step S5).

More particularly, firstly, the control unit 61 obtains the scope ID from the memory unit 241 via the signal line 216. Then, the control unit 61 refers to the associated information stored in the memory unit 62, and obtains the camera parameters (the pixel pitch δ, the baseline length b, and the focal length f) associated to that scope ID. Then, according to the obtained camera parameters (the pixel pitch δ, the baseline length b, and the focal length f) and according to the positions of the two marks 201 and 202, which are included in the main image F1 as well as the right-side image, as detected at Step S4; the control unit 61 calculates a three-dimensional position (X1, Y1, Z1) of the mark 201 and a three-dimensional position (X2, Y2, Z2) of the mark 202 according to Equations 1, 4, and 5. More particularly, the three-dimensional position (X1, Y1, Z1) of the mark 201 and the three-dimensional position (X2, Y2, Z2) of the mark 202 are expressed according to Equations 6 to 11 given below.

$$X 1 = \frac{b \cdot u 1 L}{( u 1 L - u 1 R )} \qquad ( 6 )$$

$$Y 1 = \frac{b \cdot v 1 L}{( u 1 L - u 1 R )} \qquad ( 7 )$$

$$Z 1 = \frac{fb}{δ ( u 1 L - u 1 R )} \qquad ( 8 )$$

$$X 2 = \frac{b \cdot u 2 L}{( u 2 L - u 2 R )} \qquad ( 9 )$$

$$Y 2 = \frac{b \cdot v 2 L}{( u 2 L - u 2 R )} \qquad ( 10 )$$

$$Z 2 = \frac{fb}{δ ( u 2 L - u 2 R )} \qquad ( 11 )$$

After the operation at Step S5 is performed, based on the three-dimensional position (X1, Y1, Z1) of the mark 201 and the three-dimensional position (X2, Y2, Z2) of the mark 202 as calculated at Step S5, the control unit 61 calculates a current baseline length b′ in the stereo camera 213 (Step S6).

Herein, assume that a parallax d1 represents the parallax (u1L-u1R) of the mark 201, and a parallax d2 represents the parallax (u2R-u2R) of the mark 202. Then, a distance Dist between the two marks 201 and 202 is calculated as given below in Equation 12.

$$\begin{matrix}{Dist}^{2} = {( X 1 - X 2 )}^{2} + {( Y 1 - Y 2 )}^{2} + {( Z 1 - Z 2 )}^{2} \\ = {{b}^{2} ( \frac{u 1 L}{d 1} - \frac{u 2 L}{d 2} )}^{2} + {{b}^{2} ( \frac{v 1 L}{d 1} - \frac{v 2 L}{d 2} )}^{2} + \frac{{f}^{2} {b}^{2}}{{δ}^{2}} {( \frac{1}{d 1} - \frac{1}{d 2} )}^{2}\end{matrix} \qquad ( 12 )$$

Moreover, Equation 12 is converted into Equation 13 given below.

$${b}^{2} = \frac{{Dist}^{2}}{{( \frac{u 1 L}{d 1} - \frac{u 2 L}{d 2} )}^{2} + {( \frac{v 1 L}{d 1} - \frac{v 2 L}{d 2} )}^{2} + \frac{{r}^{2}}{{δ}^{2}} ( \frac{1}{d 1} - \frac{1}{d 2} )} \qquad ( 13 )$$

Since the distance between the two marks 201 and 202 is the known distance D, the control unit 61 substitutes the distance D for the distance Dist in Equation 13 and calculates the current baseline length b′. More particularly, the baseline length b′ is expressed according to Equation 14 given below.

$${b}^{{′}^{2}} = \frac{{D}^{2}}{{( \frac{u 1 L}{d 1} - \frac{u 2 L}{d 2} )}^{2} + {( \frac{v 1 L}{d 1} - \frac{v 2 L}{d 2} )}^{2} + \frac{{r}^{2}}{{δ}^{2}} {( \frac{1}{d 1} - \frac{1}{d 2} )}^{2}} \qquad ( 14 )$$

After the operation at Step S6 is performed, the control unit 61 substitutes the baseline length b, which represents one of the camera parameters obtained at Step S5, with the baseline length b′ calculated at Step S6; and calibrates that camera parameter (Step S7).

Meanwhile, it is not limited to substitute the baseline length b with the baseline length b′. Alternatively, the shift in the baseline length (b′−b=Δb) can be saved in an overwriting manner as a camera parameter, and accordingly that camera parameter can be calibrated.

According to the present embodiment described above, the following effects are achieved.

In the control device 6 according to the present embodiment, the control unit 61 generates the calibration image F by superimposing the imaging guides G1 and G2 onto the main image F1 that represents a stereo image. Then, the control unit 61 calibrates the camera parameters of she stereo camera 213 based on the stereo image (the main image F1 and the right-side image) that is taken by the stereo camera 213 in the state in which the two marks 201 and 202 in the calibration image F have fallen into a specific positional relationship with the imaging guides G1 and G2.

The specific positional relationship indicates the positioning of the two marks 201 and 202 within the imaging guide G1. That is, as compared to a mark having a low imaged height (i.e., a mark close to the optical axis), a mark having a high imaged height (i.e., a mark at a distance from the optical axis) gets more easily affected by the distortion. Hence, as a result of maintaining the abovementioned positional relationship, the imaged heights of the two marks 201 and 202 can be lowered and the effect of distortion can be held down. That enables highly accurate calibration of the camera parameters.

Moreover, the specific positional relationship indicates the positioning of the outer edge of the chart 200 in between the imaging guides G1 and G2. That is, as compared to a mark having a smaller depth (i.e., a mark close to the stereo camera 213), a mark having a greater depth (i.e., a mark at a distance from the stereo camera 213) is more prone to the occurrence of an error in its measurement position. Hence, as a result of maintaining the abovementioned positional relationship, the depths of the two marks 201 and 202 can be appropriately set, thereby enabling highly accurate calibration of the camera parameters.

According to the explanation given above, in the control device 6 according to the present embodiment, it becomes possible to provide support for calibrating the camera parameters with a high degree of accuracy.

Moreover, in the control device 6 according to the present embodiment, as far as the calibration of the camera parameters of the stereo camera 213 is concerned, the control unit 61 calibrates only the baseline length. Hence, the calibration of the camera parameters can be appropriately performed with the bare minimum processing.

### Other Embodiments

Till now, an illustrative embodiment of the disclosure was described. However, the present embodiment is not to be limited by the embodiment described above.

### FIRST MODIFICATION EXAMPLE

FIG. 13 is a diagram illustrating a first modification example of the embodiment.

In the embodiment described above, the shape of the two marks 201 and 202 is not limited to a circular shape. Alternatively, the two marks 201 and 202 can have a shape as illustrated in FIG. 13 according to the first modification example.

According to the first modification example, as illustrated in FIG. 13, of a checkered pattern made of two white quadrangles 2011 and 2012 (or 2021 and 2022) and two black quadrangles, the mark 201 (the mark 202) is formed using the two white quadrangles 2011 and 2012 (two white quadrangles 2021 and 2022). Meanwhile, in FIG. 13, the region other than the marks 201 and 202 is black in color. Hence, the two black quadrangles are not distinguishable from that region.

At Step S4, the control unit 61 detects the point of intersection of the two white quadrangles 2011 and 2012 (the two white quadrangles 2021 and 2022) as the position of the mark 201 (the mark 202) in the main image F1 as well as the right-side image.

### SECOND MODIFICATION EXAMPLE

FIG. 14 is a diagram illustrating a second modification example of the embodiment.

In the second modification example, the shape of the two marks 201 and 202 is not limited to a circular shape. Alternatively, the marks 201 and 201 can be quadrangular according to the second modification example as illustrated in FIG. 14.

At Step S4, in an identical manner to the embodiment described above, the control unit 61 detects the centroid positions of the marks 201 and 202, which are included in the main image F1 and the right-side image, as the positions of the marks 201 and 202.

### THIRD MODIFICATION EXAMPLE

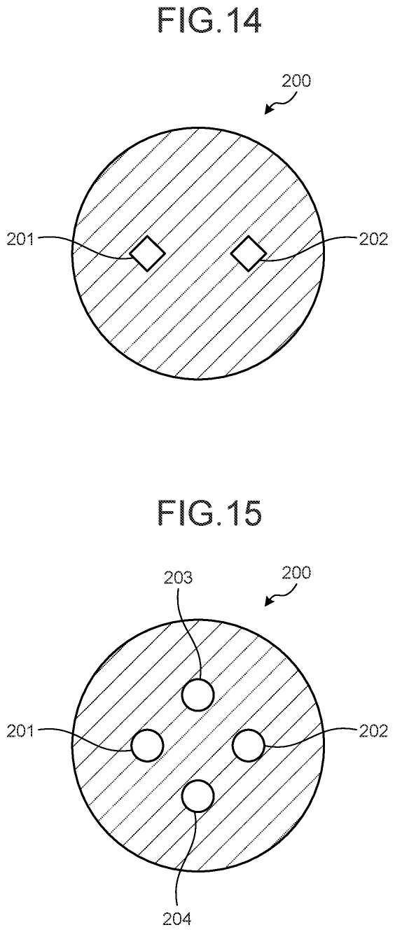

FIG. 15 is a diagram illustrating a third modification example of the embodiment.

In the embodiment described above, although there are two marks 201 and 202 formed in the chart 200, the number of marks is not limited to two and can be equal to or greater than three. For example, in the third modification example illustrated in FIG. 15, four marks 201 to 204 are formed in the chart 200.

With such a configuration, at Step S4, in case there is failure in the detection of the positions of the marks 201 and 202 that are included in the main image F1 and the right-side image, the other marks 203 and 204 can be used instead. Meanwhile, in order to calibrate the camera parameters, either all of the marks 201 to 204 can be used, or only the two marks separated by the maximum distance in the horizontal direction (in the example illustrated in FIG. 15, the marks 201 and 202) can be used. Moreover, if three or more marks are set in the chart 200; then, as compared to the configuration having only two marks 201 and 202, at the time of pressing the release button 221 at Step S3, the operator operates the insertion portion 21 while confirming the calibration image F, thereby making it easier to set the state in which the three or more marks are positioned within the imaging guide G1.

### FOURTH MODIFICATION EXAMPLE

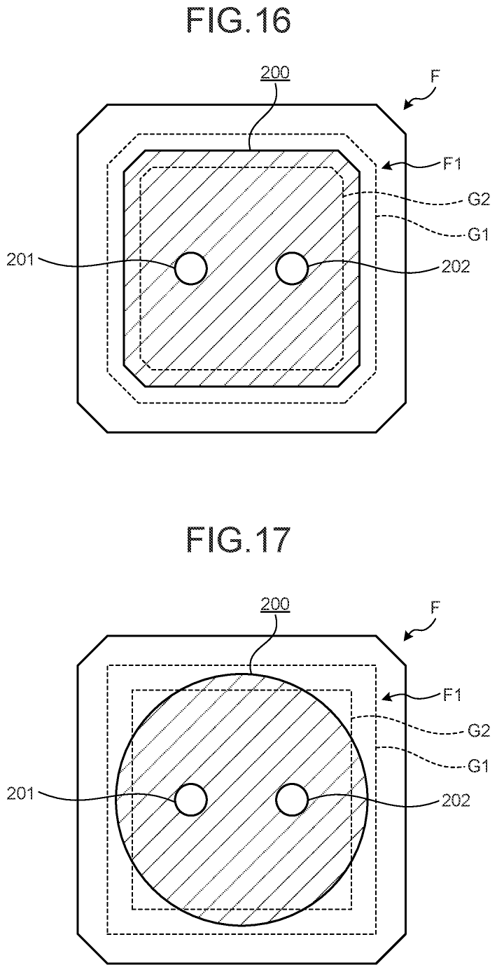

FIG. 16 is a diagram illustrating a fourth modification example of the embodiment.

In the embodiment described above, the shape of the outer edge of the chart 200 is not limited to a circular shape, and can be of any other shape. For example, in the fourth modification example illustrated in FIG. 16, corresponding to the octagonal field of view in the endoscope 2 (in FIG. 16, the outermost solid line), the outer edge of the chart 200 is also set to be octagonal in shape.

In an identical manner, in the embodiment described above, the shape of the imaging guides G1 and G2 is not limited to a circular shape, and can be of any other shape. For example, in the fourth modification example illustrated in FIG. 16, corresponding to the shape of the outer edge of the chart 200, the imaging guides G1 and G2 are set to be octagonal in shape.

### FIFTH MODIFICATION EXAMPLE

FIG. 17 is a diagram illustrating a fifth modification example of the embodiment.

In the embodiment described above, the shape of the imaging guides G1 and G2 is not limited to a circular shape, and can be a square shape as illustrated in FIG. 17 according to the fifth modification example. Then, at the time of pressing the release button 221 at Step S3, the operator operates the insertion portion 21 while confirming the calibration image F, so that the state is set in which the two marks 201 and 202 are positioned within the imaging guide G1 and in which four points of the outer edge of the chart 200 are positioned in between the imaging, guides G1 and G2. As a result, the two marks 201 and 202 fail into the specific positional relationship according to the disclosure with respect to the imaging guides G1 and G2.

### SIXTH MODIFICATION EXAMPLE

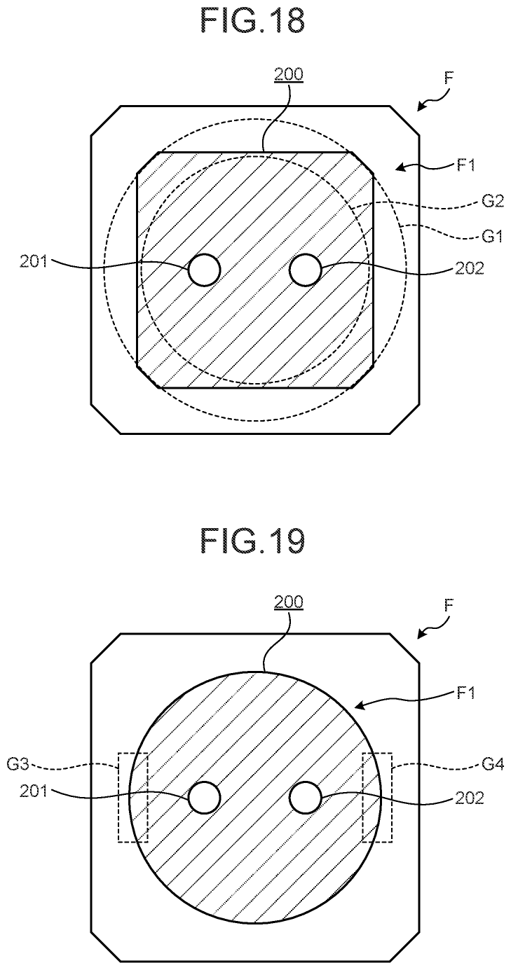

FIG. 18 is a diagram illustrating a sixth modification example of the embodiment.

In the embodiment described above, the shape of the outer edge of the chart 200 is not limited to a circular shape, and can be of any other shape. For example, in the sixth modification example illustrated in FIG. 18, corresponding to the octagonal field of view of the endoscope 2 (in FIG. 18, the outermost solid line), the outer edge of the chart 200 is also set to be octagonal in shape. Then, at the time of pressing the release button 221 at Step S3, the operator operates the insertion portion 21 while confirming the calibration image F, so that the state is set in which the two marks 201 and 202 are positioned within the imaging guide G1 and in which four corner portions of the chart 200 are positioned in between the imaging guides G1 and G2. As a result, the two marks 201 and 202 fall into the specific positional relationship according to the disclosure with respect to the imaging guides G1 and G2.

### Seventh Embodiment

FIG. 19 is a diagram illustrating a seventh modification example of the embodiment.

In the embodiment described above, the imaging guides according to the disclosure are not limited to the imaging guides G1 and G2 explained in the embodiment, and alternatively imaging guides G3 and G4 according to the seventh modification example illustrated in FIG. 19 can be adopted.

As illustrated in FIG. 19, the imaging guides G3 and G4 have a rectangular shape, and are placed in a bilaterally symmetric manner around the center position of the octagonal field of view in the endoscope 2 (i.e., in FIG. 19, the outermost solid line). The imaging guides G3 and G4 not only function as the guides for the positioning of the two marks 201 and 202 in the central region of the calibration image F, but also function as the guides for ensuring that the two marks 201 and 202 are separated by a specific distance in the calibration image F. Then, at the time of pressing the release button 221 at Step S3, the operator operates the insertion portion 21 while confirming the calibration image F, so that the state is set in which the two marks 201 and 202 are positioned in between the imaging guides G3 and G4 and in which two opposite points on the outer edge of the chart 200 are positioned within the imaging guides G3 and G4, respectively. As a result, the two marks 201 and 202 fall into the specific positional relationship according to the disclosure with respect to the imaging guides G3 and G4. Meanwhile, in FIG. 19, although the imaging guides G3 and G4 are placed in a bilaterally symmetric manner, they can alternatively be placed in the vertical direction.

### EIGHTH MODIFICATION EXAMPLE

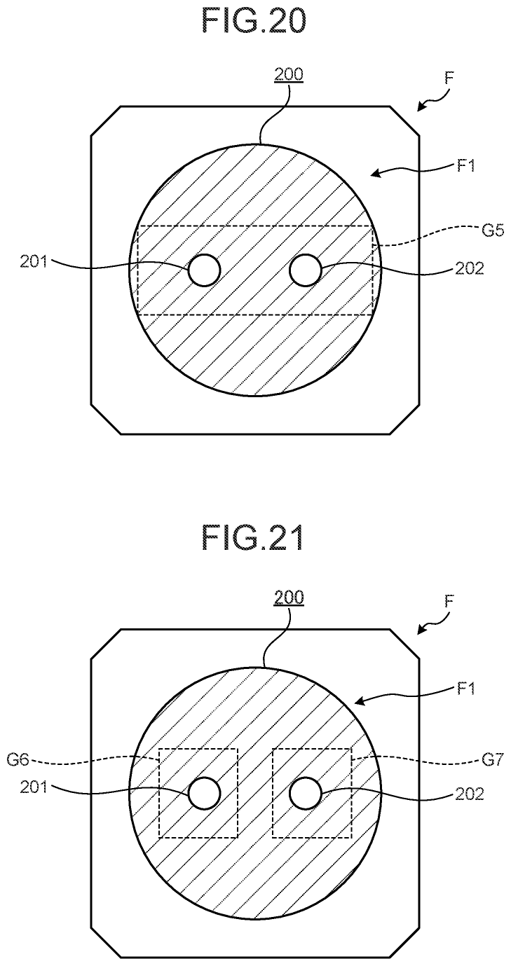

FIG. 20 is a diagram illustrating an eighth modification example of the embodiment.

In the embodiment described above, the imaging guides according to the disclosure are not limited to the imaging guides G1 and G2 explained in the embodiment, and alternatively an imaging guide G5 according to the eighth modification example illustrated in FIG. 20 can be adopted.

As illustrated in FIG. 20, the imaging guide G5 has a rectangular shape extending in the right-left direction, and is placed in such a way that the center position of the octagonal field of view in the endoscope 2 (i.e., in FIG. 20, the outermost solid line) serves as the center of the rectangle. The imaging guide G5 not only functions as the guide for the positioning of the two marks 201 and 202 in the central region of the calibration image F, but also functions as the guide for ensuring that the two marks 201 and 202 are separated by a specific distance in the calibration image F. Then, at the time of pressing the release button 221 at Step S3, the operator operates the insertion portion 21 while confirming the calibration image F, so that the state is set in which the two marks 201 and 202 are positioned within the imaging guide G5 and in which the imaging guide G5 is positioned on the inside of the outer edge of the chart 200. As a result, the two marks 201 and 202 fall into the specific positional relationship according to the disclosure with respect to the imaging guide G5.

### NINTH MODIFICATION EXAMPLE

FIG. 21 is a diagram illustrating a ninth modification example of the embodiment.

In the embodiment described above, the imaging guides according to the disclosure are not limited to the imaging guides G1 and G2 explained in the embodiment, and alternatively imaging guides G6 and G7 according to the ninth modification example illustrated in FIG. 21 can be adopted.

As illustrated in FIG. 21, the imaging guides G6 and G7 have a rectangular shape, and are placed in a bilaterally symmetric manner around the center position of the octagonal field of view in the endoscope 2 (i.e., in FIG. 21, the outermost solid line). The imaging guides G6 and G7 not only function as the guides for the positioning of the two marks 201 and 202 in the central region of the calibration image F, but also function as the guides for ensuring that the two marks 201 and 202 are separated by a specific distance in the calibration image F. Then, at the time or pressing the release button 221 at Step S3, the operator operates the insertion portion 21 while confirming the calibration image F, so that the state is set in which the two marks 201 and 202 are positioned within the imaging guides G6 and G7, respectively. As a result, the two marks 201 and 202 fall into the specific positional relationship according to the disclosure with respect to the imaging guides G6 and G7.

### 10-TH MODIFICATION EXAMPLE

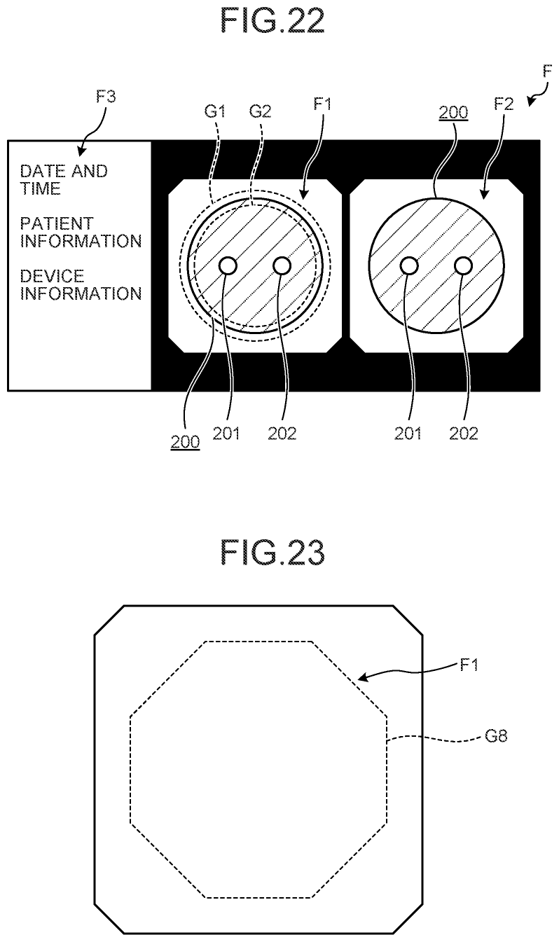

FIG. 22 is a diagram illustrating a 10-th modification example of the embodiment.

In the embodiment described above, the calibration image according to the disclosure is not limited to the calibration image F. Alternatively, the calibration image F according to the 10-th modification example illustrated in FIG. 22 can be adopted.

As illustrated in FIG. 22, the calibration image F according to the 10-th modification example includes an image formed as a result of superimposing the imaging guides G1 and G2 onto the main image F1; includes a right-side image F2; and includes a textual information display region F3. The right-side image F2 is taken by the stereo camera 213 at the same time of taking the main image F1. In the textual information display region F3, textual information such as date and time, patient information, and device information is displayed.

### 11-TH MODIFICATION EXAMPLE

FIG. 23 is a diagram illustrating an 11-th modification example of the embodiment.

In the embodiment described above, when the endoscope system 1 is switched to the observation mode, the control unit 61 can superimpose a measurable region G8 illustrated in FIG. 23 onto the main image F1. As a result, in the observation mode, image formed by superimposing the measurable region G8 onto the main image F1 is displayed in the display device 3.

As illustrated in FIG. 23, the measurable region G8 has an octagonal shape and represents a region in which the three-dimensional position of the photographic subject 100 can be measured in an effective manner.

However, the shape of the measurable region G8 is not limited to the octagonal shape, and can be of any other shape such as a circular shape and a rectangular shape.

### 12-TH MODIFICATION EXAMPLE

In the embodiment described above, at the time of obtaining the main image F1 and the right-side image at Step S3, the operator presses the release button 221. However, that is not the only possible case.

For example, at Step S3, the control unit 61. determines whether or not the two marks 201 and 202 in the main image F1 have fallen into a specific positional relationship with reference to the imaging guides G1 and G2. In other words, the control unit 61 determines whether or not the two marks 201 and 202 are positioned within the imaging guide G1 and whether or not the outer edge of the chart 200 is positioned in between the imaging guides G1 and G2. Then, the control unit 61 obtains the stereo image (the left-side image and the right-side image) that is taken by the stereo camera 213 at the point of time when the determination is affirmative.

With such a configuration, the operator is no more required to press the release button 221, thereby enabling achieving enhancement in the user-friendliness. Meanwhile, since the operations according to the 12-th modification example are performed internally in the control unit 61, they are not limited to be performed with respect to the main image F1, and can also be performed with respect to the right-side image, which is not displayed, using the information about the imaging guides G1 and G2.

### 13-TH MODIFICATION EXAMPLE

In the embodiment described above, the camera parameters are stored in the control device 6 (the memory unit 62). However, that is not the only possible case. Alternatively, the camera parameters can be stored in the endoscope 2 (the memory unit 241). That is, at the time of calculating the three-dimensional positions or the two marks 201 and 202 at Step S5, the control unit 61 obtains the camera parameters (the pixel pitch δ, the baseline length b, and the focal length f) from the memory unit 241. Moreover, at Step S7, the control unit 61 calibrates the camera parameters stored in the memory unit 241.

According to the calibration device, the endoscope system, the calibration method, and the computer program product according to the disclosure, it becomes possible to provide support for calibrating the camera parameters with a high degree of accuracy.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A calibration device comprising a processor configured to process a stereo image which is taken by a stereo camera disposed in front end portion of an endoscope, the processor being configured to
superimpose an imaging guide onto the stereo image to generate a calibration image that is to be displayed in a display, and
calibrate a camera parameter of the stereo camera based on the stereo image taken by the stereo camera in state in which a plurality of marks included in a chart representing a photographic subject image in the calibration image fall into a specific positional relationship with respect to the imaging guide.

2. The calibration device according to claim 1, wherein the processor is configured to
detect positions of the plurality of marks in the stereo image,
calculate three-dimensional positions of the plurality of marks based on the positions of the plurality of marks in the stereo image,
calculate a baseline length of the stereo camera based on the three-dimensional positions of the plurality of marks and based on an actual distance among the plurality of marks, and
calibrate, according to the calculated baseline length, a parameter that is included in the camera parameter and that is related to the baseline length.

3. The calibration device according to claim 1, wherein the processor is configured to determine whether or not the plurality of marks in the calibration image have fallen into the specific positional relationship with respect to the imaging guide.

4. The calibration device according to claim 1, wherein
the imaging guide represents a guide for guiding positioning of the plurality of marks in a central region of the calibration image, and
when positioned in the central region, the plurality of marks in the calibration image fall into the specific positional relationship with respect to the imaging guide.

5. The calibration device according to claim 1, wherein
the imaging guide represents a guide for ensuring that the plurality of marks in the calibration image are separated by a specific gap, and
when set to be separated by the specific gap, the plurality of marks in the calibration image fall into the specific positional relationship with respect to the imaging guide.

6. An endoscope system comprising:
an endoscope that includes a stereo camera installed at front end portion of the endoscope;
a calibration device that includes a processor configured to process a stereo image taken by the stereo camera; and
a display configured to display an image, the processor being configured to
superimpose an imaging guide onto the stereo image to generate a calibration image that is to be displayed in the display, and
calibrate a camera parameter of the stereo camera based on the stereo image taken by the stereo camera in state in which a plurality of marks included in a chart representing a photographic subject image in the calibration image fall into a specific positional relationship with respect to the imaging guide.

7. The endoscope system according to claim 6, wherein the processor is configured to switch between
a calibration mode in which the calibration image is generated and a camera parameter is calibrated, and
an observation mode in which an operation of the endoscope is controlled for observing inside of a subject.

8. The endoscope system according to claim 6, wherein the processor is configured to
determine whether or not the plurality of marks in the calibration image have fallen into the specific position relationship with respect to the imaging guide,
obtain the stereo image that is taken by the stereo camera at point of time when it is determined that the plurality of marks in the calibration image have fallen into the specific position relationship with respect to the imaging guide, and
calibrate the camera parameter of the stereo camera based on the obtained stereo image.

9. The endoscope system according to claim 6, wherein
the endoscope further includes a memory configured to store the camera parameter, and
the processor is configured to calibrate the camera parameter stored in the memory.

10. A calibration method implemented by a processor of a calibration device, the calibration method comprising:
superimposing an imaging guide onto a stereo image which is taken by a stereo camera disposed in front end portion of an endoscope to generate a calibration image that is to be displayed in a display; and
calibrating a camera parameter of the stereo camera based on the stereo image taken by the stereo camera in state in which a plurality of marks included in a chart representing a photographic subject image in the calibration image fall into a specific positional relationship with respect to the imaging guide.

11. A non-transitory computer-readable recording medium with an executable program stored thereon, the program causing a processor of a calibration device to execute:
superimposing an imaging guide onto a stereo image which is taken by a stereo camera disposed in front end portion of an endoscope to generate a calibration image that is to be displayed in a display; and
calibrating a camera parameter of the stereo camera based on the stereo image taken by the stereo camera in state in which a plurality of marks included in a chart representing a photographic subject image in the calibration image fall into a specific positional relationship with respect to the imaging guide.
